(19) United States
(12) Patent Application Publication
(10) Pub. No.: US 2007/0212326 A1
(43) Pub. Date: Sep. 13, 2007

(54) POLYQUATERNARY ORGANOSILICON COMPOUNDS-CONTAINING COMPOSITION

(75) Inventors: Christian Ochs, Burghausen (DE); Birgit Schwab, Munich (DE)

Correspondence Address: BROOKS KUSHMAN P.C., 1000 TOWN CENTER, TWENTY-SECOND FLOOR, SOUTHFIELD, MI, 48075, US

(73) Assignee: WACKER CHEMIE AG, Munich (DE)

(21) Appl. No.: 11/572,535

(22) PCT Filed: Jul. 21, 2005
(86) PCT No.: PCT/EP05/07965

§ 371 Date: Jan. 23, 2007

(30) Foreign Application Priority Data
Jul. 29, 2004 (DE) 10 2004 036 717.5

(51) Int. Cl. A61K 31/785; A61K 9/70

(52) U.S. Cl. 424/78.27; 424/443; 427/2.24

(57) ABSTRACT

Polymers containing repeating organopolysiloxane blocks and including quaternary ammonium groups in the polymer chain exhibit good biocidal properties, while also exhibiting good washfastness, hydrophilicity, and softness.

This invention relates to compositions containing poly-quaternary organosilicon compounds, their production and their use for antimicrobial finishing of surfaces.

A multiplicity of industries have an appreciable need for a durably antimicrobial finish for surfaces and articles. Hitherto it has been preferably organic or inorganic chemicals, their solutions or mixtures which have been used for this purpose. Acting as disinfectants, they are indeed more or less antimicrobial, but their effect is frequently very unspecific and not durable. Furthermore, most of the substances used are frequently toxic themselves in their pure form, or form in the course of their life cycle degradation products which are not generally recognized as not safe.

A further fundamental problem of the biocides used to date is that they have to have a certain solubility in water to be effective, which is why the biocidal finish is often only effective for a very limited period. But this also means that, once they have been applied, the active components usually do not remain at their site of application but tend to escape over time, in a more or less uncontrolled fashion, into the environment and perhaps accumulate there, which leads to problems in the long run. Recent proposals therefore seek to anchor the antimicrobially active group in a polymer matrix. Such antimicrobially active polymers and their formulations should by virtue of the much reduced migratability of the active component not only be environmentally compatible on a sustained basis but ideally even be non-toxic for higher organisms and also durably permanent.

The good antimicrobial efficacy of formulations of trialkoxysilane compounds having quaternary nitrogen sites has been known since the early 1970s and is well documented in the literature. An immense disadvantage of this widely used class of compounds is that they are solids because of their ionic character. Handling and metering solids, however, is generally more costly and inconvenient than in the case of liquids, so that most users favor application from solution or liquid formulations. Many of the solvents used, examples being short-chain alcohols, various glycol ethers, ethyl acetate, methyl ethyl ketone or weakly polar hydrocarbons, such as THF and toluene, however, have a high vapor pressure and also low boiling and flash points, which, in the processing facilities, leads not only to immense safety-engineering problems but also to a health and environmental impact that is no longer acceptable. Alternatively, an application from water was proposed in the past. But studies have shown that the trialkoxysilane group is hydrolyzed in aqueous media and is essentially present therein as a trihydroxysilyl group which is prone to condensation (U.S. Pat. No. 6,469,120 B1). Aqueous solutions of trialkoxysilanes having quaternary nitrogen sites therefore require careful, costly and inconvenient stabilization, have long-term stability in storage in high dilution only, and can only be used within a narrow pH range. In addition, apart from antimicrobial properties, they endow the finished materials with no further silicone-typical benefits.

Organopolysiloxanes having quaternary nitrogen groups have long been known from the literature. Their formulations are for example widely used as textile auxiliaries (WO 03/066708 A1), as a constituent of cosmetic composition (WO 01/41719 A1, WO 41720 A1, WO 41721 A1), as an ingredient in laundry detergent formulations (EP 1199350 B1) and also household cleaners (EP 1133545 B1). Any antimicrobial activity of polysiloxanes containing quat groups and of the formulations of these polysiloxanes has hitherto not been extensively documented, and close study of the data reveals in all cases that the allegedly biocidal properties have merely been deduced from antibacterial activity against an extremely small number of bacteria. Formulations of quat-containing polysiloxanes having a genuine antimicrobial broadband action against a multiplicity of different microorganisms, including fungi, bacteria, yeasts and algae for example, are therefore desirable.

Amphiphilic siloxane block and random siloxane copolymers having 3-(trialkylammoniumpropyl) side groups are described by Sauvet et al. in J. Polym. Science, Part A: Polymer Chemistry, 2003, 41, 2939-2948 in a comparative study. The polymers exhibit good bactericidal properties against E. coli and S. aureus, apparently independently of the distribution of the quaternary nitrogen sites over the overall molecule. However, no action is described against other bacterial strains or against fungi, yeasts and algae.

The picture is similar for the polydimethylsiloxanes having 3-(N-octyldimethylammonium)propyl side groups investigated in J. Appl. Polym. Science 2000, 75, 1005-1012 (Sauvet et al.). Here too the effect is limited to exclusively antibacterial activity against some selective strains (E. coli, A. hydrophila, P. aeruginosa). Furthermore, as with the previously cited paper, the focus is exclusively on the bactericidal properties of the polymers without any silicone-typical benefit-effects which might be obtainable with the disclosed compounds or their formulations, being discussed.

U.S. Pat. No. 6,384,254 B1 discloses quaternary nitrogen group-containing polysiloxanes and formulations thereof for antibacterial finishing of fibers or fibrous products. The compositions described provide fibers and textiles with a silicone-typical softness as well as bactericidal properties. In addition, the finish possesses a certain degree of durability over a few wash cycles. Furthermore, the investigations on antibacterial activity are merely restricted to S. aureus as sole bacterial strain.

All previously described antibacterial organopolysiloxanes are moreover exclusively organopolysiloxanes having lateral quat groups. However, a person skilled in the art knows that organopolysiloxanes having quaternary ammonium side groups can harbor high toxicological potential. Past studies have shown that the high toxicity of such systems is influenced by the distance between two quat units in particular. For the previously described antibacterially active organopolysiloxanes having quaternary ammonium groups, a similar, toxicologically unsafe property profile cannot be absolutely ruled out, in particular since their syntheses take place via statistical condensation and equilibration reactions which may at any time result in unfavorable spacings of the lateral quat groups. As a person skilled in the art also knows, behavior generally recognized as safe by toxicologists can in the final analysis only be guaranteed in the case of α,ω-terminal systems, owing to a defined, consistent distance between two quat groups.

The present invention provides compositions containing
(A) organosilicon compounds having at least one unit of the formula
   —[R²(SiR₂O)_{b}—SiR₂—R²—N⁺R¹₂]ₙ—·n X⁻ (I)
   where
   R in each occurrence may be the same or different and is a monovalent, optionally substituted, hydrocarbyl radical which has 1 to 18 carbon atoms and may be interrupted by oxygen atoms,
   R¹ in each occurrence may be the same or different and is a monovalent, optionally substituted, hydrocarbyl radical having 1 to 18 carbon atoms or may be part of a bridging alkylene radical,
   R² is a divalent hydrocarbyl radical which has at least 2 carbon atoms and which contains at least one hydroxyl group and/or is interrupted by one or more oxygen atoms and/or attached to silicon via oxygen,
   X⁻ is an organic or inorganic anion,
   b is an integer of at least 1 and
   n is an integer of at least 1,
(B) at least one solvent selected from the group consisting of
   (B1) water,
   (B2) organosiloxanes other than component (A), or
   (B3) polar organic solvents having an electrical dipole moment of >1 debye (20° C.), optionally
   (C) surface-active agents and optionally
   (D) further materials.

Depending on the identity of the organosilicon compounds (A), the compositions of the present invention can be present as solutions or in the form of dispersions, such as a micro- or macroemulsion for example.

The compositions of the present invention contain the organosilicon compounds (A) in amounts of preferably 10⁻⁵% to 99% by weight, more preferably in the range from 0.01% to 90% by weight and even more preferably in the range from 0.01% to 50% by weight, all based on the total weight of the compositions of the present invention.

The organosilicon compounds (A) used according to the present invention can be any desired organosilicon compounds having at least one unit of the formula (I), in which case they can be not only pure siloxanes, i.e., ≡Si—O—Si≡ structures, but also silcarbanes, i.e., ≡Si—R′—Si≡ structures where R′ is a divalent, optionally substituted and/or heteroatom-interrupted hydrocarbyl radical, or any desired copolymers comprising organosilicon groups.

Examples of R radicals are alkyl radicals, such as methyl, ethyl, n-propyl, isopropyl, 1-n-butyl, 2-n-butyl, isobutyl, tert-butyl, n-pentyl, isopentyl, neopentyl, tert-pentyl; hexyl radicals, such as n-hexyl; heptyl radicals, such as n-heptyl; octyl radicals, such as n-octyl and isooctyl radicals, such as 2,2,4-trimethylpentyl; nonyl radicals, such as n-nonyl; decyl radicals, such as n-decyl; dodecyl radicals such as n-dodecyl; octadecyl radicals, such as n-octadecyl; cycloalkyl radicals, such as cyclopentyl, cyclohexyl, cycloheptyl and methylcyclohexyl; alkenyl radicals, such as vinyl, 1-propenyl and 2-propenyl; aryl radicals, such as phenyl, naphthyl, anthryl and phenanthryl; alkaryl radicals, such as o-, m-, p-tolyl radicals; xylyl radicals and ethylphenyl radicals; and aralkyl radicals, such as benzyl, α-phenylethyl and β-phenylethyl.

Examples of substituted R radicals are methoxyethyl, ethoxyethyl and (2-ethoxy)ethoxyethyl.

Preferably, R comprises hydrocarbyl radicals having 1 to 12 carbon atoms which are optionally substituted with halogen atoms, amino groups, ether groups, ester groups, epoxy groups, mercapto groups, cyano groups or (poly)glycol radicals, the latter being constructed from oxyethylene and/or oxypropylene units and more preferably alkyl radicals having 1 to 6 carbon atoms; and especially methyl.

Examples of R¹ radicals include the examples indicated for R.

Preferably, R¹ comprises hydrocarbyl radicals having 1 to 18 carbon atoms, more preferably alkyl radicals having 1 to 8 carbon atoms and benzyl radicals. However, R¹ can also be a divalent radical derived therefrom, so that for example two R¹ radicals combine with the nitrogen atom to form a ring. When the R¹ radical comprises substituted hydrocarbyl radicals, hydroxyl groups are preferred as substituents.

Examples of the X⁻ anion are organic anions, such as carboxylate ions, enolate ions and sulfonate ions, and also inorganic anions, such as halide ions, for example fluoride ions, chloride ions, bromide ions and iodide ions, and sulfate ions.

Preferably, the X⁻ anion comprises carboxylate ions and halide ions, more preferably chloride ions and acetate ions.

Examples of R² are divalent, linear, cyclic or branched, saturated or unsaturated hydrocarbyl radicals which are substituted with hydroxyl groups and/or interrupted by one or more oxygen atoms and/or attached to silicon via oxygen, examples being the radicals

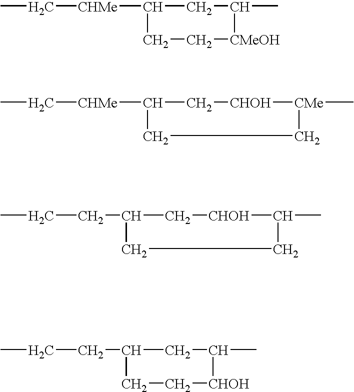

—(CH₂)₂O—, —(CH₂)₃O—, —(CH₂)₃OCH₂—, —(CH₂)₃OCH₂—CH(OH)—CH₂— and —(CH₂)₃OCH₂—CH[—CH₂(OH)]—, where Me is methyl.

Preferably R² comprises —(CH₂)₃OCH₂—, —(CH₂)₃OCH₂—CH(OH)—CH₂— and —(CH₂)₃OCH₂—CH[—CH₂(OH)]—.

Preferably, b comprises an integer from 1 to 5000, more preferably from 2 to 500.

Preferably, n comprises an integer from 1 to 100, more preferably from 1 to 75 and in particular from 2 to 50.

Preferably, the organosilicon compounds (A) used according to the present invention are those of the general formula
D¹ₐ-[R²(SiR₂O)_{b}—SiR₂—R²—N⁺R¹₂]ₙ-D²ₐ·nX⁻ (II)

where
D¹ in each occurrence may be the same or different and is a hydrogen atom, a hydroxyl radical, a halide radical, an epoxy-functional radical, an —NR*₂ radical or a monovalent organic radical, where R* in each occurrence may be the same or different and is a hydrogen atom or a monovalent, optionally substituted hydrocarbyl radical and the —NR*₂ radical may also be present as an ammonium salt, and
D² is a group of the formula
   —R²—(SiR₂O)_{b}—SiR₂—R²-D¹ (III)
and
a is 0 or 1,
while R, R¹, R², X⁻, b and n are each as defined above.

Examples of organic radical D¹ are alkyl radicals and alkoxy radicals, of halide radicals —Cl and —Br, of epoxy-functional radicals the radical

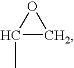

nitrogenous organic radicals, such as amines, sulfur-containing organic radicals, such as sulfonate radicals, and organic or inorganic anions added onto carbon, such as for example carboxylates and halogenated hydrocarbyl radicals. An example of the —NR*₂ radical is —N(CH₃)₂.

Preferably, D¹ comprises hydrogen, hydroxyl, halide, alkyl, alkoxy, epoxy-functional radicals, carboxylate, enolate or the —NR*₂ radical where R* is as defined above, more preferably D¹ comprises hydrogen, hydroxyl, alkoxy, halide, the radical

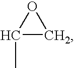

the acetate or propionate radical and also the radical —NR*₂.

Examples of R* are hydrogen atom and the examples specified above for R.

R* comprises preferably hydrogen or a hydrocarbyl radical having 1 to 18 carbon atoms, more preferably an alkyl radical having 1 to 8 carbon atoms, especially preferably methyl and benzyl. But R* can also be a divalent radical derived therefrom, so that for example two R* radicals combine with the nitrogen atom to form a ring. When R* comprises substituted hydrocarbyl radicals, hydroxyl groups are preferred as substituents.

The organosilicon compounds (A) of the formula (II) which are used according to the present invention can comprise cyclic compounds, i.e., where a is 0, and also linear compounds where a is in each case 1.

Preferably, a is 1.

More preferably, the organosilicon compounds (A) which are used according to the present invention comprise linear polymers of the formula (II) where a is 1, R² is —(CH₂)₃OCH₂—, —(CH₂)₃OCH₂—CH(OH)—CH₂— or —(CH₂)₃OCH₂—CH[—CH₂(OH)]— and D¹ is —Cl, —N(CH₃)₂,

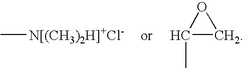

Examples of the organosilicon compound (A) which are used according to the present invention are

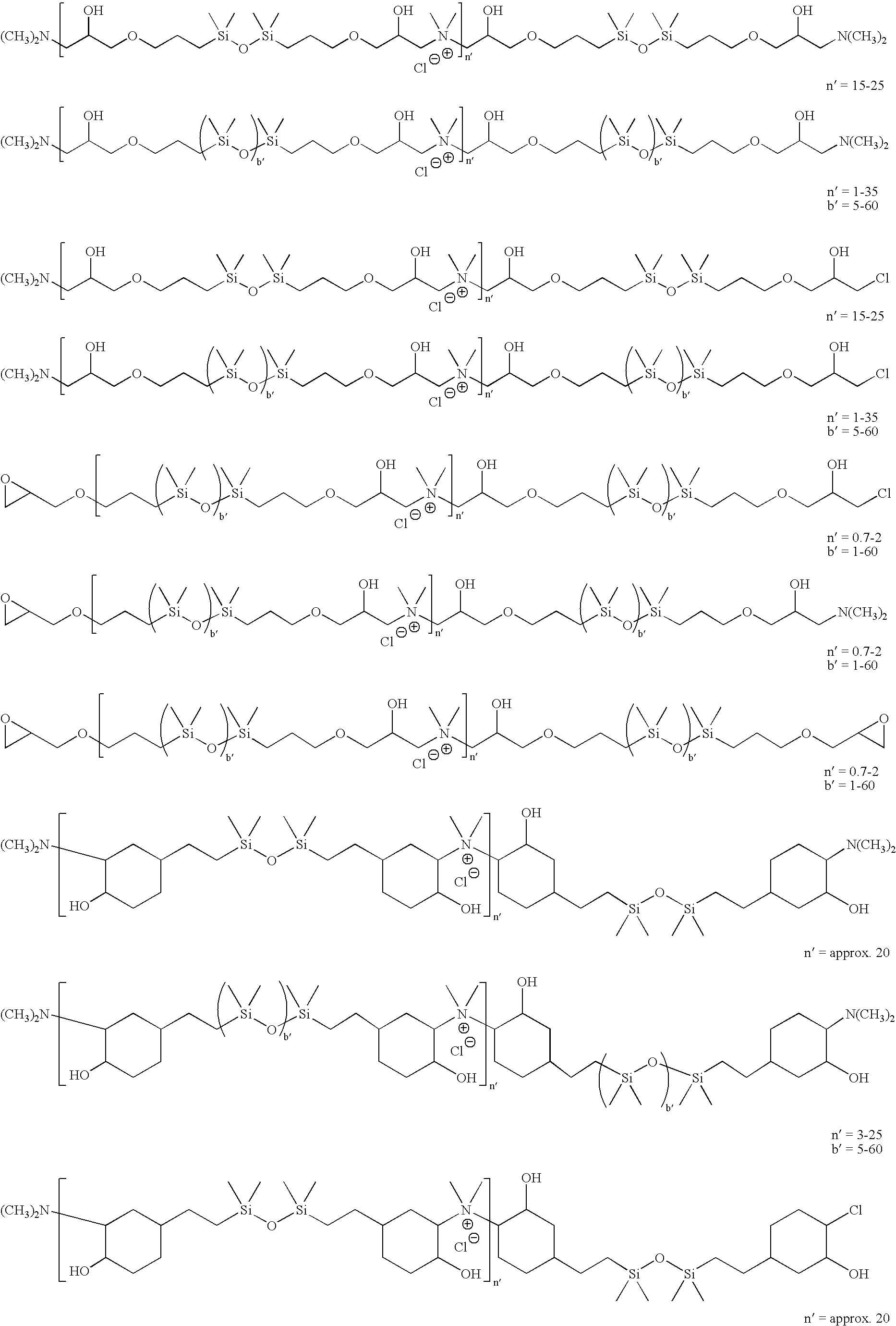

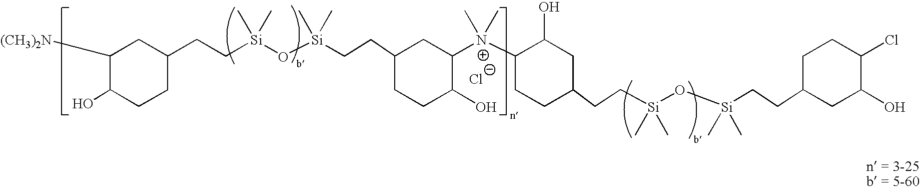

where the —Cl and —N(CH₃)₂ substituents on the cyclohexyl radical can independently occupy not only the 4 position but also the 3 position relative to the —CH₂CH₂— group, and the values stated for the indices n′ and b′ are to be understood as ranges for polymeric compounds of very broad molar mass distribution, and also the examples mentioned in U.S. Pat. No. 6,730,766 at column 3 line 58 to column 4 line 36, which shall count as part of the disclosure content of the present invention.

The organosilicon compounds (A) which are used according to the present invention have a viscosity of preferably 10³ to 10⁸ mpas and more preferably 10⁴ to 5*10⁷ mpas, all at 25° C.

The organosilicon compounds (A) which are used according to the present invention are commercially available products and/or obtainable according to known processes, for example by reaction of the corresponding epoxy-functional silanes and/or siloxanes with dialkylammonium salts such as for example dimethylammonium chloride or by reaction of the corresponding amino compounds with alkyl halides.

The term “solvent” herein is not to be understood as meaning that all the components have to dissolve therein.

The solvents (B) which are used according to the present invention preferably comprise water (B1) or polar organic solvents (B3) having an electric dipole moment of >1 debye (20° C.) and more preferably comprise water, monohydric alcohols or polyhydric alcohols.

Any desired water can be used as solvent (B1), in which case solvent (B1) can contain further materials which occur naturally in water, examples being minerals, bacteria, trace elements, dissolved gases, suspended matter, etc., or which can typically be added for water applications or for achieving particular effects.

Examples of solvents (B1) are natural waters, for example rainwater, groundwater, spring water, river water and sea water, chemical waters, for example completely ion-free water, distilled or (repeatedly) redistilled water, water for medical or pharmaceutical purposes, for example purified water (Aqua purificata; Pharm. Eur. 3), Aqua deionisata, Aqua destillata, Aqua bidestillata, Aqua ad injectionam or Aqua conservata, drinking water in accordance with the German Drinking Water Regulations, and mineral waters.

Solvent (B1) preferably comprises drinking water in accordance with the German Drinking Water Regulations, completely ion-free water, distilled water and purified water (Aqua purificata) and more preferably comprises completely ion-free water, distilled water and purified water (Aqua purificata).

Examples of organosiloxanes useful as solvent (B) include linear or cyclic organopolysiloxanes having alkyl radicals which are optionally substituted with amino, hydroxyl, polyether or carboxyl groups, examples being
cyclic siloxanes consisting of 3 to 8 diorganosiloxy units, for example octamethyltetracyclosiloxane,
trimethylsiloxy-, alkoxydimethylsiloxy- or hydroxydimethylsiloxy-terminated, amino-functional polysiloxanes having lateral and/or terminal —CH₂—NH₂, —(CH₂)₃—NH₂ or —(CH₂)₃—NH—(CH₂)₂—NH₂ groups and an amine number of 0.5 to 11.5, the amine number being the number of ml of 1N HCl needed to neutralize 1 g of substance, examples being Me₃SiO—(SiMe₂O)₉₅—{SiMe[(CH₂)₃—NH—(CH₂)₂—NH₂]O}₅—SiMe₃,
carbinol-functional polysiloxanes having lateral and/or terminal —(CH₂)₃—OH or —(CH₂)₂—OH groups and a carbinol group content of at least 3% by weight, examples being Me₃SiO—(SiMe₂O)₃₀—{SiMe[(CH₂)₃—OH]O}₃₀—SiMe₃,
polyether-functional polysiloxanes having lateral and/or terminal polyether groups and a polyether content of at least 10% by weight, examples being Me₃SiO—(SiMe₂O)₇₀—{SiMe[(CH₂)₃—O—(C₂H₄O)₂₅—(C₃H₆O)₂₅H]O}₅—SiMe₃ or
carboxyl-functional polysiloxanes having lateral and/or terminal —(CH₂)₂—COOH, —(CH₂)₁₀—COOH or —(CH₂)₂—CH(COOH)—CH₂—COOH groups and a carboxyl group content of at least 3% by weight, examples being Me₃SiO—(SiMe₂O)₃₀—{SiMe—[(CH₂)₂—CH(COOH)−CH₂—COOH]O}₃₀—SiMe₃.

The term “organopolysiloxanes” herein shall also comprise dimeric siloxanes as well as polymeric and oligomeric siloxanes.

When the solvent (B) which is used according to the present invention comprises organosiloxanes, cyclic siloxanes consisting of 3 to 8 diorganosiloxy units, the aforementioned amino-functional polysiloxanes, polyether-functional siloxanes and carboxyl-functional polysiloxanes are preferred, with cyclic siloxanes consisting of 3 to 8 diorganosiloxy units being particularly preferred.

The organosiloxanes (B2) preferably comprise siloxanes which are waxily solid at 20° C. and a pressure of 900 to 1100 hPa, or liquid siloxanes having a viscosity of 0.5 to 10 000 000 mm²/s (25° C.), with liquid siloxanes being particularly preferred.

When the siloxanes (B2) comprise liquid siloxanes, liquid siloxanes having a viscosity of 0.5 to 100 000 mm²/s are preferred and those having a viscosity of 0.5 to 1000 mm²/s are particularly preferred, all at 25° C.

Examples of polar organic solvents (B3) which are used according to the present invention are monohydric or polyhydric alcohols such as for example methanol, ethanol, n-propanol, i-propanol, 1,2-propanediol, 1,3-propanediol, 1-butanol, 2-butanol, tert-butanol, 1,4-butanediol, 1-pentanol, 2-pentanol, 3-pentanol, 1,5-pentanediol, 1-hexanol, cyclohexanol, 1-heptanol, 1-octanol, 1-decanol, lauryl alcohol, myristyl alcohol, stearyl alcohol, benzyl alcohol, diethylene glycol, triethylene glycol, dipropylene glycol, ethylene glycol monomethyl ether, ethylene glycol monoethyl ether, ethylene glycol monobutyl ether, diethylene glycol monomethyl ether and diethylene glycol monobutyl ether, aliphatically saturated polyethers, such as polyethylene glycol, polypropylene glycol, poly-THF and their inter-polymers, monomethyl, monoethyl and monobutyl ethers and monoacyl esters of aliphatically saturated polyethers, linear or cyclic ethers, examples being diethyl ether, methyl tert-butyl ether, ethylene glycol dimethyl ether, diethylene glycol diethyl ether, tetrahydrofuran or dioxane, linear or cyclic ketones, examples being acetone or diisopropyl ketone, carboxylic acids, for example formic acid, acetic acid or propionic acid, carboxylic esters, for example methyl acetate, ethyl acetate or butyl acetate, linear or cyclic carbonates, for example dimethyl carbonate or ethylene carbonate, chlorinated hydrocarbons, for example methylene chloride, chloroform, 1,2-dichloroethane or chlorobenzene, aprotic polar solvents, for example acetonitrile, acetamide, dimethylformamide, tetrahydro-1,3-dimethyl-2(1H) -pyrimidinone (DMPU), hexamethylphosphoramide (HMPT), dimethyl sulfoxide (DMSO), sulfolane or CO₂, and also organic ionic liquids, for example 1,3-dimethylimidazolium methosulfate or 1-butyl-4-methylpyridinium chloride.

When polar organic solvents (B3) are used as solvent (B), the I type composition according to the present invention preferably comprises component (A) dissolved in component (B).

When water (B1) is used as polar solvent (B), the II type compositions of the present invention preferably comprise, in particular according to the nature of the organosilicon compounds (A) used, an aqueous solution or an aqueous dispersion.

When polar organosiloxanes (B2) are used as solvent (B), the III type compositions of the present invention preferably comprise solutions or dispersions, and in the last case mentioned it is preferably the siloxanes (B2) which constitute the continuous phase.

When nonpolar organosiloxanes (B2) are used as solvent (B), the IV type compositions of the present invention preferably comprise dispersions, and in the case mentioned last it is preferably the siloxanes (B2) which constitute the continuous phase.

The compositions of the present invention contain solvent (B) in amounts of preferably 1% to 99% by weight and more preferably 10% to 90% by weight, all based on the total weight of the composition of the present invention.

Examples of surface-active agents (C) optionally used according to the present invention include any desired surface-active agents, for example emulsifiers previously also used to prepare dispersions for example. Component (C) can be used here not only in pure form but also as solutions of one or more kinds of component (C) in water or organic solvents.

Examples of suitable nonionic emulsifiers (C) include sorbitan esters of fatty acids having 10 to 22 carbon atoms, polyoxyethylene sorbitan esters of fatty acids having 10 to 22 carbon atoms and up to 35% by weight of ethylene oxide content, for example the ethylene oxide condensates of sorbitan monolaurate, of sorbitan monomyristate, of sorbitan monostearate, of sorbitan tristearate or of sorbitan trioleate, polyoxyethylene derivatives of phenols having 6 to 20 carbon atoms on the aromatic moiety and up to 95% by weight ethylene oxide content, for example the ethylene oxide condensates of dodecylphenol, of myristylphenol, of octylphenol or of stearylphenol, polyoxyethylene condensates of fatty acids or fatty alcohols having 8 to 22 carbon atoms with up to 95% by weight ethylene oxide content, for example the ethylene oxide condensates of lauryl alcohol, of stearyl alcohol or of isotridecyl alcohol, ethylene oxide condensates of fatty acid monoesters of glycerol having 10 to 22 carbon atoms and up to 95% by weight of ethylene oxide; mono- or diethanolamides of fatty acids having 10 to 22 carbon atoms, fatty imidazolines having 6 to 20 carbon atoms, for example cocoimidazoline, cetylimidazoline, 1-hydroxyethyl-2-heptadecenylimidazoline or cocosulfoimidazoline, polyvinyl alcohols obtainable by saponification of polyvinyl acetate, and also phosphate esters.

Examples of suitable anionic emulsifiers (C) include alkylarylsulfonates having 6 to 20 carbon atoms in the alkyl group, for example sodium dodecylbenzenesulfonate or potassium dodecylbenzenesulfonate, fatty sulfates having 8 to 22 carbon atoms, for example sodium dodecylsulfate, potassium dodecylsulfate, triethanolammonium dodecylsulfate, sodium stearylsulfate, potassium stearylsulfate, triethanolammonium stearylsulfate, alkylsulfonates having 10 to 22 carbon atoms, for example sodium dodecylsulfonate, potassium dodecylsulfonate, sodium stearylsulfonate or potassium stearylsulfonate, fatty acid soaps having 8 to 22 carbon atoms, for example trimethyldodecylammonium chloride, sodium laurate, sodium myristate or potassium myristate, alkali metal salts of dialkylsulfosuccinates, and also alkali metal salts of carboxylated, ethoxylated alcohols having 10 to 22 carbon atoms and up to 95 percent of ethylene oxide.

Examples of cationic emulsifiers (C) are organic fatty ammonium compounds having 10 to 22 carbon atoms, for example trimethylstearylammonium methosulfate, and fatty morpholine oxides having 10 to 22 carbon atoms.

Examples of amphiphilic emulsifiers (C) are fatty aminobetaines and amidobetaines having 10 to 22 carbon atoms, for example decylaminobetaine, fatty amidosulfobetaines having 10 to 22 carbon atoms, for example cocoamidosulfobetaine or olylamidobetaine, and also fatty amine oxides having 10 to 22 carbon atoms, for example n-cocomorpholine oxide, decyldimethylamine oxide and cocoamidodimethylamine oxide.

Examples of inorganic solids which can likewise be used as emulsifiers (C) are finely divided silicas or bentonites, as described for example in U.S. Pat. No. 6,605,351 or DE 19742759 A.

Preferably, the surface-active agents (C) which are used according to the present invention comprise nonionic, cationic, or anionic emulsifiers or inorganic solid emulsifiers, particular preference being given to nonionic or anionic emulsifiers.

The compositions of the present invention preferably contain component (C) when component (A) does not completely dissolve in component (B), for example when nonpolar siloxane (B2) is used as solvent (B). On the other hand, the use of component (C) is redundant when component (A) itself has emulsifier properties.

When the compositions of the present invention contain surface-active agents (C), these are used in amounts of preferably 0.1 to 60 parts by weight and more preferably 1 to 40 parts by weight, all based on 100 parts by weight of organosilicon compound (A).

The compositions of the present invention can further contain any desired auxiliary or filler materials (D), for example agents to standardize the pH, such as basic materials or inorganic acids, catalysts, defoamers, foam stabilizers, rheology regulators, thickeners, dyes, pigments, opacifiers, flame retardants, redox stabilizers, antioxidants, light stabilizers, heat stabilizers, odorants, odor-inhibiting or odor-reducing materials, natural substances, for example plant or fruit extracts, and also inorganic or organic polymers, for example finely divided silica.

Preferably, component (D) optionally used in the composition of the present invention comprises catalysts, agents to standardize the pH, dyes, pigments, opacifiers, flame retardants, redox stabilizers, antioxidants, light stabilizers, heat stabilizers, odorants, odor-inhibiting or odor-reducing materials, and also inorganic or organic polymers, for example finely divided silica.

When the compositions of the present invention contain further materials (D), these are used in amounts of preferably 0.01 to 100 parts by weight and more preferably 1 to 50 parts by weight, all based on 100 parts by weight of organosilicon compound (A).

The components used according to the present invention can in each case be one kind of such a component and also a mixture of at least two kinds of a particular component.

The compositions of the present invention have a solids content of preferably 0.1% to 90% by weight, or preferably 1% to 70% by weight and especially 1% to 50% by weight.

The term “solids content” is herein to be understood as meaning the sum total of components (A) and if appropriate (C) and if appropriate (D).

The compositions of the present invention have a pH of preferably 2 to 12 and more preferably 4 to 10, all at 25° C.

Preferably, the present invention's compositions of the I type are those containing

(A) organosilicon compounds having at least one unit of the formula (I),

(B) polar organic solvents having an electrical dipole moment of >1 debye (20° C.) and optionally

(D) further materials.

More preferably, the present invention's compositions of the I type are those consisting of

(A) organosilicon compounds of the formula (II),

(B) mono- and polyhydric alcohols and optionally

(D) further materials.

The present invention's compositions of the I type have a viscosity of preferably up to 100 000 mm²/s and more preferably 1 to 10 000 mm²/s, all at 25° C.

Preferably, the present invention's compositions of the II type are those containing

(A) organosilicon compounds having at least one unit of the formula (I),

(B) water, optionally

(C) surface-active agents and optionally

(D) further materials.

More preferably, the present invention's compositions of the II type are those consisting of

(A) organosilicon compounds of the formula (II),

(B) water, optionally

(C) surface-active agents and optionally

(D) further materials.

The present invention's compositions of the II type have a viscosity of preferably up to 100 000 mm²/s and more preferably 1 to 10 000 mm²/s, all at 25° C., in the case of solutions.

The present invention's compositions of the II type have a viscosity of preferably up to 10 000 mm²/s and more preferably 1 to 1000 mm²/s, all at 25° C., in the case of dispersions.

Preferably, the present invention's compositions of the III type are those containing

(A) organosilicon compounds having at least one unit of the formula (I),

(B) polar siloxanes, optionally

(C) surface-active agents and optionally

(D) further materials.

More preferably, the present invention's compositions of the III type are those consisting of

(A) organosilicon compounds of the formula (II),

(B) polar siloxanes, optionally

(C) surface-active agents and optionally

(D) further materials.

The present invention's compositions of the III type have a viscosity of preferably 0.5 to 100 000 mm²/s and more preferably 1 to 10 000 mm²/s, all at 25° C., in the case of solutions.

The present invention's compositions of the III type have a viscosity of preferably up to 10 000 mm²/s and more preferably 1 to 2000 mm²/s, all at 25° C., in the case of dispersions.

Preferably, the present invention's compositions of the IV type are those containing

(A) organosilicon compounds having at least one unit of the formula (I),

(B) nonpolar siloxanes, optionally

(C) surface-active agents and optionally

(D) further materials.

More preferably, the present invention's compositions of the IV type are those consisting of

(A) organosilicon compounds of the formula (II),

(B) nonpolar siloxanes,

(C) surface-active agents and optionally

(D) further materials.

The present invention's compositions of the IV type have a viscosity of preferably up to 10 000 mm²/s and more preferably 1 to 1000 mm²/s, all at 25° C., in the case of dispersions.

To produce the compositions of the present invention, all the constituents can in principle be mixed with one another in any desired order irrespective of the particular type. Mixing can take place at room temperature and the pressure of the ambient atmosphere, i.e., about 900 to 1100 hPa, in accordance with any desired and hitherto known processes. If desired, however, mixing can also take place at higher temperatures, for example at temperatures in the range from 30 to 200° C.

Of course, component (A) can also be prepared in situ, and used without isolation or further workup steps, in the production of the composition of the present invention.

When the compositions of the present invention comprise dispersions, these may be obtained according to any desired, previously known processes for producing emulsions and dispersions, wherein preferably first component (A) is dispersed with component (B) and if appropriate (C) and subsequently the component (D), if used, is added.

The compositions of the present invention have the advantage that they are easy to produce, have a very high stability in storage and also provide surface finishes which do not yellow and which exhibit a biocidal effect over a long period.

The compositions of the present invention further have the advantage that they are highly active antimicrobially.

The compositions of the present invention further have the advantage that they are not toxic to humans. By suitable combination of siloxane building block and quat group density in the organosilicon compound (A) used according to the present invention, the compositions of the present invention can be made as formulations which are highly antibacterial even at very low concentration and have good environmental compatibility and low toxic potential.

It is a further advantage that the organosilicon compounds (A) used in the compositions of the present invention, as well as a hydroxyl group, can have further different functional groups (for example epoxy, amino or chloroalkyl groups) which can be utilized for durable incorporation of the antimicrobially active polymer in organic or silicon-based polymers.

The compositions of the present invention can then be used for all purposes for which solutions or dispersions of organosilicon compounds have previously been used. The compositions of the present invention are suitable for all applications concerned with antimicrobial treatment or finish of industrial products, for example dispersions, emulsions and mixtures, in particular surfaces, and/or with the achievement of silicone-typical surface effects.

The present invention further provides a process for endowing surfaces with an antimicrobial finish, characterized in that the composition according to the present invention is applied to the surface to be treated.

Application in accordance with the present invention can be effected according to hitherto known methods as typically also used hitherto for finishing surfaces of a respective substrate. In the process of the present invention, the surface to be treated is treated with the compositions of the present invention for a time sufficient for finishing. This can be effected for example by coating, spraying, brushing, doctoring, padding or exhausting the formulations of the present invention onto the substrate or by dipping the substrate into the compositions of the present invention and also by coextrusion or blending, in which case further process steps can follow in all cases. Alternatively, the process of the present invention can be carried out, in lieu of the present invention's compositions themselves, by using formulations containing the compositions of the present invention, as can be the case for example with household cleaner or shampoo formulations.

Suitable substrates for the treatment by the process of the present invention are those having hard or soft surfaces of any kind. Preferably they comprise natural or artificial fibers, textile wovens and knits, textile sheet materials, tissue papers and wovens, papers, skin, hairs, leather, coated surfaces or surfaces consisting of metal, glass, ceramic, glass ceramic, enamel, mineral materials, wood, cork, plastics and also artificial and natural elastomers. More preferably, the surfaces which are treated by the process of the present invention comprise textiles, tissue papers, skin, coated surfaces, metallic surfaces, glass, ceramic, mineral materials, wood, plastics and elastomers.

The process of the present invention can be used in all sectors concerned with achieving an antimicrobial treatment or finish of surfaces, for example in the case of surfaces which are exposed to the weather, in the case of surfaces and articles in the household and food sectors, for example floors, tiles, windows, refrigerators, freezers, ovens, toys, baby and children's articles, packaging, pipelines, containers or filters, surfaces and articles in the care sector (nursing care, intensive care, infant care or care for old people) and clinical sector (hospitals, rooms for medical treatments or interventions, isolation wards), medical articles or products, for example wound dressings, tubes, sterile filters or transplants, surfaces and articles in the hygiene and sanitary sector, for example toilets, toothbrushes, shower booths or curtains, medical applications, for example use as disinfectants, and also the antifouling sector.

Further examples of applications for the process of the present invention where the object is to achieve a combination of antimicrobial finish with further, advantageous surface effects are building, building protection and leather applications (for example the antimicrobial finishes of exteriors, walls, joining compounds or building materials, or leather in combination with hydrophobicization), textile and tissue applications (for example the antimicrobial finishing of yarns, fibers, textiles, wovens, papers or the like in combination with softening, hydrophilicization, antistatic properties and improved affinity), cosmetic applications, in particular from the hair care and skin care sector (for example the combination of an antimicrobial effect with improved affinity, improved hand and hair luster, reduced electrostatic charge buildup on hair, reduced combing force, the general protection of keratinic fibers against splitting, drying out and structure-damaging environmental effects, a pleasant skin feel, a reduced stickiness for the cosmetic formulation, a reduced tendency of pigments or fillers to aggregate, and also the formation of a hydrophobic but breathable barrier which can lead for example to an improved water fastness on the part of the cosmetic product) and also polish and home care applications (for example the antimicrobial finishing of surfaces in combination with gloss enhancement, reduced water film drying time, improved affinity and product formulability).

The present invention's process for finishing surfaces is carried out at temperatures of preferably −100 to +300° C. and more preferably −30 to +200° C. and the pressure of the ambient atmosphere, i.e., about 900 to 1100 hPa. However, higher or lower pressures can also be employed, if desired.

The process of the present invention has the advantage that surfaces of any kind can be rendered antimicrobial, with the antimicrobial properties being durable if appropriate. Advantageously, the antimicrobialness in question is a genuine biocidal effect which extends over a very broad spectrum and to a multiplicity of organisms, such as gram+ and gram− bacteria, fungi, yeasts and algae. The biocidal effect and also the effect limit, moreover, can be set in a specific manner by simply varying the siloxane building block in the organosilicon compound (A) used according to the present invention, the active component always being polymer bound and hence constituting a low bioavailability to higher organisms.

The process of the present invention further has the advantage that it leads to a multiplicity of additional, partly permanent, surface effects which hitherto have only been achievable through the combined use of two or more products. Thus, the finished surfaces can be uniquely endowed with, as well as antimicrobial properties, further positive properties, for example softness, hydrophilicity, antistatic finishing, improved affinity in finishing operations, reduced combing force, accelerated surface drying, luster and so on. For example, the formulations of the present invention and/or the process of the present invention make it possible to achieve very good softening of cellulosic textile or tissue wovens combined with high hydrophilicity and resistance to microbes. It is similarly possible, using the formulations of the present invention and/or the process of the present invention, to render for example polyester-based fibers and wovens simultaneously semipermanently soft, hydrophilic, antimicrobial and antistatic.

The process of the present invention further has the advantage that it produces a surface finish which does not yellow and which exhibits a biocidal effect over a long period.

In the examples described hereinbelow, all viscosities relate to a temperature of 25° C. Unless otherwise stated, the examples hereinbelow are carried out at a pressure of the ambient atmosphere, i.e., at 1000 hPa, say, and at room temperature, i.e., at about 23° C., or at a temperature which results automatically when the components are added together at room temperature without additional heating or cooling. Furthermore, all parts and percentages are by weight, unless otherwise stated.

The examples utilize the following abbreviations:

emulsifier A: isotridecyl alcohol polyethylene oxide ether, about 10 ethylene oxide units, 85% in water (commercially available under the designation of Lutensol® TO 109 from BASF AG, Germany);

emulsifier B: ammonium lauryl sulfate, 40% in water (commercially available under the designation of Texapon® A from Cognis, Germany);

emulsifier C: isotridecyl alcohol polyethylene oxide ether, about 5 ethylene oxide units (commercially available under the designation of Lutensol® TO 5 from BASF AG, Germany).

Testing for antimicrobial activity was carried out as follows:

Four gram+ and three gram− bacterial strains, six fungal strains, two yeast strains and one algal strain were used (table 1). All the microorganisms used are commercially available. The production of the requisite media, nutrient solutions and agar plates, or the growth and cultivation of the microorganisms, were carried out using microbiological standard procedures in a conventional manner. Before testing, the inventive compositions used were sterilized by autoclaving at 120° C. (30 minutes) to prevent any cross contamination with extraneous organisms.

TABLE 1

| | | | Incubation |
|---|---|---|---|
| | Strain | Order | temperature |
| A | Bacillus subtilis | gram+ | 30° C. |
| B | Micrococcus luteus | bacteria | 30° C. |
| C | Corynebacterium glutamicum | | 30° C. |
| D | Staphylococcus aureus | | 30° C. |
| E | Pseudomonas aeruginosa | gram− | 30° C. |
| F | Escherichia coli | bacteria | 37° C. |
| G | Klebsiella oxytoca | | 30° C. |
| H | Trametes versicolor | fungi | room temperature |
| I | Aspergillus niger | | room temperature |
| J | Penicillium funiculosum | | room temperature |
| K | Paecilomyces varioti Bainier | | room temperature |
| L | Gliocladium virens Miller | | room temperature |
| | et al. | | |
| M | Chaetomium globosum Kunze | | room temperature |
| N | Candida albicans | yeast | 30° C. |
| O | Pichia pastoris | | 30° C. |
| P | Desmodesmus subspicatus | alga | room temperature |

To test for antimicrobial activity, the masterplate of the microorganism in question has a single colony or a mycelium taken from it by means of a sterile inoculating loop that is streaked by means of the loop onto an agar plate. A small amount of the autoclaved inventive composition is then applied perpendicularly thereto, resulting in a cross-shaped arrangement of inoculating streak and composition to be tested. The plates are incubated at the temperature typical for the particular test strain until in that part of the inoculating streak which is not covered by the inventive composition distinct growth is visible. The extent of microbial inhibition can be estimated visually from the difference between the growth in the bare part of the inoculating streak and the part of the inoculating streak which is situated underneath or directly alongside the inventive composition.

### EXAMPLE 1

286.4 g of dimethylammonium chloride are dissolved in 1000 ml of water, 1200 g of 1,3-bis(3-glycidoxypropyl)-1,1,3,3-tetramethyldisiloxane are added, and the mixture is refluxed with thorough stirring. The reaction mixture is stirred at 105-110° C. for 2 hours, in the course of which the reaction batch changes from colorlessly turbid to a clear yellow. ¹H NMR spectroscopy confirms the formation of a polyquaternary polysiloxane having about on average 18 to 20 repeat units conforming to the formula

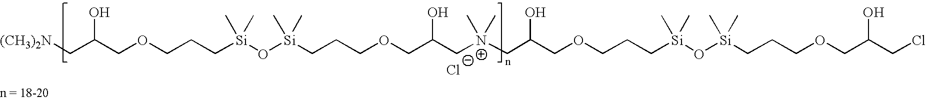

The aqueous solution thus obtained has a solids content of about 60% by weight and a viscosity of 890 mm²/s (composition of example 1).

The antimicrobial effect of the resulting composition of example 1 was investigated. The results are to be found in table 2.

TABLE 2

| Strain | Control | Example 1 | Example 2 | Example 3 |
|---|---|---|---|---|
| A | − | ++ | ++ | ++ |
| B | − | ++ | ++ | ++ |
| C | − | ++ | + | − |
| D | − | ++ | ++ | ++ |
| E | − | + | + | − |
| F | − | ++ | + | − |
| G | − | ++ | + | − |
| H | − | ++ | + | + |
| I | − | ++ | ++ | +/− |
| J | − | ++ | ++ | ++ |
| K | − | ++ | ++ | − |
| L | − | ++ | + | − |
| M | − | ++ | ++ | ++ |
| N | − | + | ++ | − |
| O | − | + | ++ | ++ |
| P | − | ++ | ++ | + |

++ very strong inhibition of microbial growth, partly with inhibitory zone
+ strong inhibition
+/− weak inhibition
− no inhibition

As table 2 reveals, the composition of example 1 has strongly antimicrobial properties which is very pronounced in strong dilution, as the following investigation concerning the effectiveness of antimicrobial inhibition shows.

Effectiveness of Antimicrobial Inhibition:

The above-prepared composition of example 1 was diluted by addition of further water to the solids content reported in table 3 (dilute composition of example 1). Then, the test for antimicrobial activity was carried out in the manner described above.

TABLE 3

| | Solids content [% by weight] | | | | | | | | | |
|---|---|---|---|---|---|---|---|---|---|---|
| Strain | 10 | 5 | 2.5 | 1 | 0.5 | 0.4 | 0.3 | 0.2 | 0.1 | 0.05 |
| A | ++ | ++ | ++ | ++ | ++ | ++ | + | +/− | − | − |
| B | ++ | ++ | ++ | ++ | ++ | ++ | ++ | ++ | + | + |
| C | ++ | ++ | + | + | + | + | + | − | − | − |
| D | ++ | ++ | ++ | ++ | ++ | + | + | + | − | − |
| E | + | + | + | + | +/− | − | − | − | − | − |
| F | ++ | ++ | + | + | + | + | + | + | + | + |
| G | ++ | + | + | + | + | +/− | − | − | − | − |
| H | ++ | + | + | + | + | + | + | +/− | − | − |
| I | ++ | + | + | +/− | − | − | − | − | − | − |
| J | ++ | + | + | + | + | + | + | +/− | − | − |
| K | ++ | + | +/− | − | − | − | − | − | − | − |
| L | ++ | + | + | +/− | − | − | − | − | − | − |
| M | + | + | + | + | − | − | − | − | − | − |
| N | + | + | + | + | +/− | − | − | − | − | − |
| O | + | + | + | + | + | + | +/− | − | − | − |
| P | ++ | ++ | + | + | + | + | + | + | + | + |

++ very strong inhibition of microbial growth, partly with inhibitory zone
+ strong inhibition
+/− weak inhibition
− no inhibition

As table 3 reveals, the dilute compositions of example 1 exhibit strong antimicrobial activity even in low concentration. The growth of gram+ bacteria is inhibited from a concentration of about 0.2% by weight, the effect limit is about 0.5% by weight for grambacteria and yeasts and about 1% by weight in the case of fungi. In selected individual cases and in the case of algae, however, antimicrobial growth is inhibited at a far lower concentration of the organopolysiloxane (B, F, G, H, J, O and P).

The minimum inhibitory concentration (MIC) of the composition of example 1 with regard to E. coli (B) and M. luteus (F) was determined in a conventional manner in the classic liquid culture test. The culture solutions are admixed with the inventive composition of example 1 in the respective concentration, inoculated with a defined number of microorganisms from a preculture (OD₆₀₀=0.01; OD₆₀₀: optical density at 600 nm) and incubated at 30° C. or 37° C. for 24 h.

Thereafter, 200 μl of each solution are plated out on agar plates, incubated for a further 24 hours, and the colonies are counted. A culture solution without addition of the composition of example 1 serves as comparative control. By correlating the number of living cells in test and comparative solutions it is possible to deduce the minimum inhibitory concentration (MIC) with regard to the respective microorganism. For the composition of example 1, the absolute MIC is 200 weight ppm for E. coli and 20 weight ppm for M. luteus.

### EXAMPLES 2 TO 4

Preparation of Siloxane I):

125 g of dimethylammonium chloride are dissolved in 900 ml of water, 1200 g of a linear siloxane consisting of dimethylsiloxy and 3-glycidoxypropyldimethylsiloxy units and having an epoxy content of 2.4 mmol/g and a viscosity of 13 mm²/S (25° C.) are added, and the mixture is heated to reflux temperature with thorough stirring. The reaction mixture is stirred at 100-110° C. for 5 hours, during which the reaction batch changes from colorlessly turbid to slightly yellow. The solvent is then removed at 120° C. under reduced pressure. The reaction product is a yellow, highly viscous oil having a viscosity of about 1 to 6×10⁶ mpas. ¹H NMR spectroscopy confirmed the formation of a polyquaternary polysiloxane having about on average 30 to 35 repeat units conforming to the formula

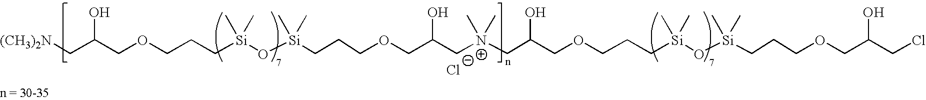

Preparation of Siloxane II):

The preparation is carried out similarly to the preparation of siloxane I), using 76 g of dimethylammonium chloride, 160 ml pf water and 1100 g of a linear siloxane consisting of dimethylsiloxy and 3-glycidoxypropyldimethylsiloxy units and having an epoxy content of 1.6 mmol/g and a viscosity of 20 mm²/s (25° C.). The reaction product is a yellow, highly viscous oil having a viscosity of about 0.5 to 2.5×10⁶ mPas. ¹H NMR spectroscopy confirms the formation of a polyquaternary polysiloxane having about on average 5 to 15 repeat units conforming to the formula

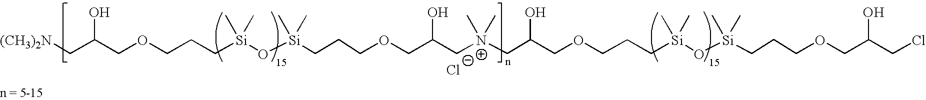

Preparation of Siloxane III):

The preparation is carried out similarly to the preparation of siloxane I) using 22.2 g of dimethylammonium chloride, 1000 g of a linear siloxane consisting of dimethylsiloxy and 3-glycidoxypropyldimethylsiloxy units and having an epoxy content of 1.6 mmol/g and a viscosity of 80 mm²/s (25° C.) and also, as solvent, 140 ml of water and 350 g of i-propanol. The reaction product is an almost colorless, viscous oil having a viscosity of about 1 to 3×10⁶ mPas. ¹H NMR spectroscopy confirmed the formation of a polyquaternary polysiloxane having about on average 3 to 5 repeat units conforming to the formula

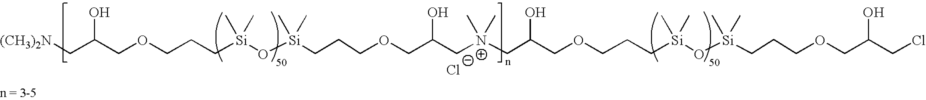

To produce the inventive compositions, the constituents mentioned in table 4 are mixed together and dispersed using an emulsifying apparatus such as an Ultra-Turrax or dissolver for example. The resulting aqueous emulsions of the high molecular weight organopolysiloxanes having quaternary nitrogen groups are further dilutable with water and have a storage life of more than 6 months at room temperature.

TABLE 4

| | Example 2 | | Example 3 | | Example 4 | |
|---|---|---|---|---|---|---|
| Quat-functional | 14.6 g of | | 24.51 g of | | 24.65 g of | |
| polysiloxane | siloxane I) | | siloxane II) | | siloxane III) | |
| Emulsifier A | 4.9 | g | 4.90 | g | 1.97 | g |
| Emulsifier B | — | | — | | 1.97 | g |
| i-Propanol | — | | — | | 1.48 | g |
| Water | 80.5 | g | 70.59 | g | 69.92 | g |
| Appearance | transparent | | creamily white | | creamily white | |
| Solids content | 18.5% | | 28.5% | | 27% | |
| Viscosity | 45 | mm²/s | 75 | mm²/s | 2.5 | mm²/s |

| | | | |
|---|---|---|---|
| Antimicrobial | good | satisfactory | none |
| effect | (cf. tab. 2) | (cf. tab. 2) | |

The antimicrobial effect of the compositions thus obtained was investigated. The results are to be found in table 2.

### EXAMPLES 5 TO 7

To produce the inventive compositions, the constituents mentioned in table 5 were mixed together and reacted in accordance with the following description:

The epoxysiloxanes used comprise in the case of

a) a linear polysiloxane consisting of dimethylsiloxy and 3-glycidoxypropyldimethylsiloxy units and having an epoxy content of 2.4 mmol/g and a viscosity of 13 mm²/s

b) a linear polysiloxane consisting of dimethylsiloxy and 3-glycidoxypropyldimethylsiloxy units and having an epoxy content of 1.6 mmol/g and a viscosity of 20 mm²/s

c) a linear polysiloxane consisting of dimethylsiloxy and 3-glycidoxypropyldimethylsiloxy units and having an epoxy content of 0.5 mmol/g and a viscosity of 80 mm²/s.

TABLE 5

| | Example 5 | | Example 6 | | Example 7 | |
|---|---|---|---|---|---|---|
| Epoxysiloxane a) | 187 | g | — | | — | |
| Epoxysiloxane b) | — | | 220 | | — | |
| Epoxysiloxane c) | — | | — | | 200 | g |
| H₂N(CH₃)₂⁺Cl⁻ | 19.4 | g | 15.2 | g | 4.4 | g |
| Water | 300 | g | 100 | g | 28 | g |
| Emulsifier C | — | | 23.5 | g | — | |
| Emulsifier A | 50 | g | 23.5 | g | 16.4 | g |
| Emulsifier B | — | | — | | 16.5 | g |
| Butyldiglycol | — | | 23.5 | g | 9.4 | g |
| i-Propanol | — | | — | | 70 | g |
| Water | 850 | g | 1200 | g | 650 | g |
| Appearance | transparent | | creamily | | creamily | |
| | | | white | | white | |
| Solids content | 18% | | 16.4% | | 24% | |
| Viscosity | 39 | mm²/s | 32 | mm²/s | 4.6 | mm²/s |

| | | | |
|---|---|---|---|
| Antimicrobial | good (like | satisfactory | none (like |
| effect | example 2) | (like example 3) | example 4) |

A solution of dimethylammonium chloride and water is mixed with the epoxysilane, emulsifier and if appropriate cosolvent, and heated to reflux temperature, by stirring. The mixture is stirred at 110° C. for 5 hours, during which the turbid starting mixture becomes clear and the viscosity increases slightly. Then, if present, the i-propanol is removed under reduced pressure, and the mixture is diluted with water to the desired solids content. The resulting aqueous emulsions of the high molecular weight organosiloxanes having quaternary nitrogen groups are further dilutable with water and have a storage life of more than 3 months at room temperate.

Antistatic Finishing and Hydrophilicization of Hydrophobic Polyester (PES) Woven and Polypropylene (PP) Nonwoven

Use was made of unfinished woven fabric polyester (PES) woven 100%, hydrophobic, and polypropylene (PP) nonwoven, hydrophobic, which were pretreatred by each being washed twice with silicone-free fully built washing powder at 95° C.

To finish, the woven samples were drenched with the respective compositions of examples 5 and 6 which had been adjusted to pH 4 with acetic acid, and squeezed off in a two-roll pad-mangle to a wet pickup of 60% (PES woven) or 85% (PP nonwoven), tentered and dried at 110° C. for 3 minutes. The fabric was then conditioned at 23° C. and 50% relative humidity for at least 12 hours.

A) Antistatic Finishing of Hydrophobic PES Woven:

The electrostatic properties of the finished PES woven were measured using an EMF 57 electro field meter from ELTEX. The chargeup voltage was 6.5 kV for all samples. The time to 50% and 90% discharge based on the respective peak value at the start of discharging was measured. As table 6 reveals, the compositions of example 5 and 6 not only have antibacterial properties but are also able to endow polyester woven with excellent antistatic properties.

TABLE 6

| | | |
|---|---|---|
| Chargeup | Peak | |
| voltage | value | Discharge time [s] |

| | [kV] | [mV] | 50% | 90% |
|---|---|---|---|---|
| Composition of | 6.5 | 170 | 0.5 | 2.5 |
| example 5 | | | | |
| Composition of | 6.5 | 180 | 9.5 | 19.6 |
| example 6 | | | | |
| Unfinished PES | 6.5 | 170 | >300 | >300 |
| woven | | | | |

B) Hydrophilicization of Hydrophobic Polyester (PES) Woven and Polypropylene (PP) Nonwoven:

Hydrophilicity was determined in a conventional manner via the droplet absorption time (time within which a drop of water applied to the woven fabric is completely absorbed by the fabric), the measurements being repeated after 5 washing cycles (wash at 40° C. with silicone-free fully built washing powder) to test durability to washing. Five determinations were performed in each case and the mean values calculated.

Compared with the commercially available textile softener according to the prior art, the composition of example 5 endows both PES woven and PP nonwoven with excellent hydrophilicity (cf. table 7). In the case of PES woven, the finish even possesses a distinct durability to washing. The washing stability on PP nonwoven is admittedly somewhat lower. Nevertheless, even on PP nonwoven there is significant retained hydrophilicity apparent after 5 washes, which is at least equal to the hydrophilicity due to the standard textile softener.

TABLE 7

| | Droplet absorption time [s] (hydrophilicity) | | | |
|---|---|---|---|---|
| | without wash | | after 5 washes | |
| | PES | PP nonwoven | PES | PP nonwoven |
| Unfinished woven | 120 | 120 | 120 | 120 |
| Composition of | 5 | 2 | 42 | 80 |
| example 5 | | | | |
| Comparative | 23 | 8 | 120 | 80 |
| formulation I* | | | | |

*Aqueous emulsion of a hydrophilic softener based on a polyether-functional aminosiloxane (obtainable from Wacker-Chemie GmbH, Germany, under the name of WETSOFT ® CTA).

### EXAMPLE 8

3.15 g of siloxane I, the preparation of which is described in example block 2-4, are thoroughly stirred together with 2.00 g of emulsifier C and adjusted with 15 g of completely ion-free water to a solids content of 25.5% by weight. A clear microemulsion is obtained.

The composition thus obtained endows terry fabric with similarly excellent softness to a commercial, classic textile softener based on aminosiloxane. In addition, however, both on terry and woven cotton fabric and also on woven CO/PES blend fabric, outstanding hydrophilicity is obtained at a level which the standard textile softener falls far short of achieving.

Compared with unfinished woven fabric, finishing the woven textile fabric with the composition obtained distinctly enhances the ease of ironing and significantly shortens ironing time.

Fabric Care Applications

By way of pretreatment, the woven test fabrics (terry towels, each 225 g; flat woven cotton fabrics, each 20×160 cm, 50 g; flat cotton/polyester (CO/PES 35/65) blend fabrics, each 15×100 cm, 45 g) were washed twice with silicone-free fully built washing powder in the full wash cycle at 95° C. and then additionally rinsed two times in the rinse cycle.

The test fabrics were finished in the rinse cycle at a German water hardness of 3°. To this end, the rinse cycle was completely traversed once, 1.5 l of drinking water, 20 g of acetic acid (100%) and also the prepared composition in example 8 being directly introduced into the washing drum before the start of the last rinse cycle. The performance tests were carried out after drying of the fabrics and conditioning at 23° C. and 60% relative humidity overnight.

A) Softness of Terry Fabric:

Softness was assessed by 10 testers, who assessed the softness of the finished terry fabrics with reference to a hand scale ranging from 0 (=very harsh) to 3 (=very soft). The hand assessment of any one sample was thus computed as the average of the points awarded to this sample by each tester.

B) Hydrophilicity of Terry, Cotton and CO/PES Blend Fabrics:

Hydrophilicity was determined in a conventional manner via the droplet absorption time.

C) Ease of Ironing of CO/PES Blend Fabrics:

Ease of ironing was assessed in a conventional manner via the time needed for a hot iron to glide down a plane, 1 m in length and inclined by 6°, onto which the fabric samples were tautly clamped.

D) Shortening of Ironing Time in the Case of Cotton Fabrics:

Ironing time is essentially determined by the number of ironing movements needed to iron a fabric site crease free. This took 13 ironing passes (100%) in the case of untreated cotton fabric and accordingly fewer in the case of the finished fabric. The computed percentage shortening of the ironing time is shown in table 8.

TABLE 8

| | Un- | WACKER ® | |
|---|---|---|---|
| | treated | Finish | Example |
| | fabric | CT 34 E* | 8 |
| Softness, terry | 0 | 3 | 2.5 |
| Droplet absorption time, terry | 1 | 80 | 2 |
| [s] (hydrophilicity) | | | |
| Droplet absorption time, cotton | 1 | 22 | 4 |
| [s] (hydrophilicity) | | | |
| Droplet absorption time, CO/PES | 6 | 76 | 16 |
| blend [s] (hydrophilicity) | | | |
| Gliding time [s] CO/PES | 18 | 3 | 5 |
| blend (ease of ironing) | | | |
| Shortening of ironing time, | — | 38% | 46% |
| cotton fabric | | | |

*Aqueous emulsion of an amino-functional polysiloxane available from Wacker-Chemie GmbH, Germany.

### EXAMPLE 9

Softening and Hydrophilicization of Tissue Papers:

Commercially available, uncoated bath tissue was used. The compositions according to examples 5, 6 and 7 were each transferred by means of a bar coater to a rubber mat and from there, by roll application using a stainless steel roll, to the tissue paper, on both sides thereof. After air drying at 23° C. and 60% relative humidity, the tissue papers were evaluated with similar active add-on (of silicone) with regard to their softness. Hand was evaluated by 10 testers who could award each paper sample 0 points (softness of A is worse than that of B), 0.5 points (softness of A is comparable to that of B) or 1 point (softness of A is superior to that of B). The results of the hand assessment are shown in table 9.

TABLE 9

| | Active | | | | | | | |
|---|---|---|---|---|---|---|---|---|
| | add-on | | | | | | | |
| | (based on | | | | | | | |
| | paper | Unfinished | Comparison | Comparison | Example | Example | Example | |
| | weight) | fabric | I* | II** | 5 | 6 | 7 | Total |
| Unfinished | — | — | 0.0 | 0.0 | 0.0 | 0.0 | 0.0 | 0.0 |
| fabric | | | | | | | | |
| Comparison | 2.4% | 1.0 | — | 1.0 | 0.0 | 0.0 | 0.5 | 2.5 |
| I* | | | | | | | | |
| Comparison | 2.5% | 1.0 | 0.0 | — | 0.0 | 0.0 | 0.5 | 1.5 |
| II** | | | | | | | | |
| Example 5 | 2.3% | 1.0 | 1.0 | 1.0 | — | 1.0 | 1.0 | 5.0 |
| Example 6 | 2.3% | 1.0 | 1.0 | 1.0 | 0.0 | — | 1.0 | 4.0 |
| Example 7 | 2.5% | 1.0 | 0.5 | 0.5 | 0.0 | 0.0 | — | 2.0 |
| *aqueous emulsion of a hydrophilic softener based on a polyether-functional aminosiloxane, available from Wacker-Chemie GmbH, Germany, under the name of WETSOFT ® CTA | | | | | | | | |
| **aqueous mixture consisting of 0.6 part of a 35% aqueous solution of a polyether-functional polysiloxane (available from Wacker-Chemie GmbH, Germany, under the name of PULPSIL ® 950 S) | | | | | | | | |
| # and 0.4 part of an aqueous emulsion of an amino-functional polysiloxane (available from Wacker-Chemie GmbH, Germany, under the name of WACKER ® FINISH CT 34 E). | | | | | | | | |

The inventive compositions have an excellent performance level with regard to the hydrophilicization of tissue paper. They are distinctly superior to the commercial standard products representing the state of the art.

1-10. (canceled)

11. A composition comprising:
(A) organosilicon compounds having at least one unit of the formula
—[R²(SiR₂O)_{b}—SiR₂—R²—N⁺R¹₂]ₙ—·nX⁻ (I)
where
R each, individually, is a monovalent, optionally substituted hydrocarbyl radical which has 1 to 18 carbon atoms and may be interrupted by oxygen atoms,
R¹ is a monovalent, optionally substituted hydrocarbyl radical having 1 to 18 carbon atoms or may be part of a bridging alkylene radical,
R² is a divalent hydrocarbyl radical which has at least 2 carbon atoms and which contains at least one hydroxyl group and/or is interrupted by one or more non-adjacent oxygen atoms and/or is attached to silicon via oxygen,
X⁻ is an organic or inorganic anion,
b is an integer of at least 1 and
n is an integer of at least 1,
(B) at least one solvent selected from the group consisting of
(B1) water,
(B2) organosiloxanes other than component (A), or
(B3) polar organic solvents having an electric dipole moment of >1 debye (20° C.), and
(C) optionally, surface-active agents.

12. The composition of claim 11, wherein the organosilicon compounds (A) have the formula
D¹ₐ-[R²(SiR₂O)_{b}—SiR₂—R²—N⁺R¹₂]ₙ-D²ₐ·nX⁻ (II)
where
D¹ each, individually is a hydrogen atom, a hydroxyl radical, a halide radical, an epoxy-functional radical, an —NR*₂ radical or a monovalent organic radical, where R* each, independently is a hydrogen atom or a monovalent, optionally substituted hydrocarbyl radical and where the —NR*₂ radical is optionally present as an ammonium salt,
D² is a group of the formula
—R²—(SiR₂O)_{b}—SiR₂—R²-D¹ (III)
and
a is 0 or 1.

13. The composition of claim 11, wherein the solvent (B) comprises a polar organic solvent (B3) having an electrical dipole moment of >1 debye (20° C.).

14. The composition of claim 13, wherein the compositions comprise:
(A) organosilicon compounds having at least one unit of the formula (I), and
(B) a polar organic solvent and having an electrical dipole moment of >1 debye (20° C.).

15. The composition of claim 11, wherein the solvent (B) comprises water (B1).

16. The composition of claim 15, wherein the compositions comprise:
(A) organosilicon compounds having at least one unit of the formula (I),
(B) water, and
(C) optionally, surface-active agents.

17. A composition of claim 11, wherein the solvent (B) comprises polar organosiloxanes (B2).

18. A composition of claim 11, wherein the solvent (B) comprises nonpolar organosiloxanes (B2).

19. A process for endowing a surface with an antimicrobial finish, comprising applying the composition of claim 11 to the surface to be treated.

20. The process of claim 19, wherein the surface treated comprise surfaces of natural or artificial fibers, textile wovens or knits, textile sheet materials, tissue papers, papers, skin, hairs, leather, coated surfaces or surfaces consisting of metal, glass, ceramic, glass ceramic, enamel, mineral materials, wood, cork, plastics, and artificial and natural elastomers.
